(19) United States
(12) Patent Application Publication
(10) Pub. No.: US 2014/0207001 A1
(43) Pub. Date: Jul. 24, 2014

(54) ULTRASONIC PROBE AND ULTRASONIC MEDICAL SYSTEM ADOPTING THE SAME

(71) Applicant: Samsung Electronics Co., Ltd., Suwon-si (KR)

(72) Inventors: Joon-ho Seo, Seoul (KR); Won-chul Bang, Seongnam-si (KR)

(73) Assignee: SAMSUNG ELECTRONICS CO., LTD., Suwon-si (KR)

(21) Appl. No.: 14/065,032

(22) Filed: Oct. 28, 2013

(30) Foreign Application Priority Data
Jan. 23, 2013 (KR) 10-2013-0007662

(51) Int. Cl. A61B 8/00; A61N 7/00

(52) U.S. Cl. CPC A61B 8/4444; A61N 7/00
USPC 600/459

(57) ABSTRACT

An ultrasonic probe and a medical system adopting the same. The ultrasonic probe includes at least one support plate having a first state of being folded and a second state of being unfolded. The ultrasonic probe also includes a plurality of ultrasonic transducers arranged on the at least one support plate.

### CROSS-REFERENCE TO RELATED APPLICATIONS

This application claims the benefit of Korean Patent Application No. 10-2013-0007662, filed on Jan. 23, 2013, in the Korean Intellectual Property Office, the entire disclosure of which is incorporated herein by reference for all purposes.

### BACKGROUND

1. Field

The following description relates to an ultrasonic probe for diagnosis and treatment, and an ultrasonic medical system adopting the same.

2. Description of the Related Art

With the development of medical research and technology, techniques for the local treatment of a tumor have evolved from invasive surgical methods, such as a laparotomy, to minimally invasive surgical methods. Further, non-invasive methods have also been developed, and a gamma knife, a cyber knife, and an ultrasonic probe have been introduced.

An ultrasonic probe uses ultrasound for the treatment of tumors by a widely-used method that is harmless to the human body and environment friendly. A high-intensity focused ultrasound (HIFU) probe is used to remove and treat an affected part. HIFU is irradiated and focused on the affected part to generate focal destruction or necrosis of tissue.

Typically, an ultrasonic probe performs treatment and/or diagnosis by externally applying ultrasonic energy to an affected part of the human body. However, gases within the digestive intestines or respiratory organs may prevent the transfer of ultrasonic energy. Thus, an external ultrasonic probe, as used from outside the human body, may have a limited treatment and/or diagnosis capacity for an affected part of the digestive intestines or respiratory organs.

### SUMMARY

In a general aspect, there is provided an ultrasonic probe including at least one support plate having a first state of being folded and a second state of being unfolded; and ultrasonic transducers arranged on the at least one support plate.

The at least one support plate may include a plurality of support plates each having a first state of being folded and a second state of being unfolded; the ultrasonic transducers may be capacitive micromachined ultrasonic transducers which are arranged in an array on at least one of the plurality of support plates; and the second states of the plurality of support plates may allow the plurality of support plates to be coupled to an inner wall of an organ.

The ultrasonic probe may further include a first member; a second member capable of moving in a lengthwise direction with respect to the first member; a plurality of first arms, each having one end connected to the first member and another end connected to one of the plurality of support plates; and a plurality of second arms, each having one end connected to the second member and another end connected to one of the plurality of first arms at a position between the one end and the another end of the plurality of first arms, wherein in response to the second member being moved with respect to the first member, the plurality of support plates are switched between the first states and the second states.

The first and second members may be tube-shaped members and at least a part of the first member may be inserted into the second member.

The ultrasonic probe may further include a rotation unit for rotating the plurality of support plates around a center axis of the ultrasonic probe.

The second member may include a fixed portion and a rotating portion that is rotatably coupled to the fixed portion and to which the plurality of first arms are connected; and the rotation unit may rotate the rotating portion.

The rotation unit may include a first gear provided at the rotating portion of the second member and having a rotation axis that is aligned with the center axis of the ultrasonic probe; a second gear provided at the fixed portion of the second member and engaged with the first gear; and a pulley provided at the fixed portion of the second member and configured to be driven by a cable to rotate the second gear.

The first and second gears may be bevel gears.

The plurality of support plates may include a first support plate on which the plurality of capacitive micromachined ultrasonic transducers are arranged and a second support plate on which the plurality of capacitive micromachined ultrasonic transducers are not arranged.

The plurality of support plates may be symmetrically arranged about the center axis of the ultrasonic probe.

The ultrasonic probe may further include a laparoscopic camera that is provided at a leading end portion of the ultrasonic probe.

In another general aspect, there is provided an ultrasonic probe including at least one support plate having a first state of being folded and a second state of being unfolded; ultrasonic transducers arranged on the at least one support plate; a first member on which the at least one support plate is supported; and a laparoscopic camera provided at a leading end portion of the first member.

The at least one support plate may include a plurality of support plates each having a first state of being folded and a second state of being unfolded; the ultrasonic transducers may be arranged in a two-dimensional plane on at least one of the plurality of support plates; and the second states of the plurality of support plates may allow the plurality of support plates to be coupled to an inner wall of an organ.

The ultrasonic probe may further include a second member capable of moving in a lengthwise direction with respect to the first member; a plurality of first arms, each having one end connected to the first member and another end connected to one of the plurality of support plates; and a plurality of second arms, each having one end connected to the second member and another end connected to one of the plurality of first arms at a position between the one end and the another end of the plurality of first arms.

The second member may include a fixed portion and a rotating portion that is rotatably coupled to the fixed portion and to which the plurality of first arms are connected; the rotating portion may be provided with a first gear; the fixed portion may be provided with a second gear that is engaged with the first gear and is provided with a pulley that is configured to be driven by a cable for rotating the second gear; and the rotating portion may be rotatable with respect to the fixed portion.

The plurality of support plates may include a first support plate on which the plurality of ultrasonic transducers are arranged and a second support plate for supporting the ultrasonic probe on an inner wall of the organ along with the first support plate.

In another general aspect, there is provided an ultrasonic medical system including the ultrasonic probe; and a controller for controlling the ultrasonic probe.

The ultrasonic medical system may further include a second member capable of moving in a lengthwise direction with respect to the first member; a plurality of first arms, each having one end connected to the first member and another end connected to one of the plurality of support plates; and a plurality of second arms, each having one end connected to the second member and another end connected to one of the plurality of first arms at a position between the one end and the another end of the plurality of first arms.

The second member may include a fixed portion and a rotating portion that is rotatably coupled to the fixed portion and to which the plurality of first arms may be connected, the rotating portion may be provided with a first gear having a rotation axis aligned with a center axis of the ultrasonic probe, the fixed portion may be provided with a second gear that is engaged with the first gear and is provided with a pulley that is configured to be driven by a cable for rotating the second gear, and the rotating portion may be rotatable with respect to the fixed portion.

The plurality of support plates may include a first support plate on which the plurality of ultrasonic transducers are arranged and a second support plate for supporting the ultrasonic probe on an inner wall of the organ along with the first support plate.

### BRIEF DESCRIPTION OF THE DRAWINGS

FIG. 1 is a diagram illustrating an example of an ultrasonic probe in which a support plate is in a folded state.

FIG. 2 is diagram illustrating an example of the ultrasonic probe of FIG. 1, in which the support plate is in an unfolded state.

FIG. 3 is a diagram illustrating an example of an ultrasonic medical system.

FIG. 4 is a diagram illustrating an example of a capacitive micromachined ultrasonic transducer (cMUT).

FIG. 5 is a diagram illustrating an example of a connection structure for folding/unfolding the support plate.

FIG. 6 is a diagram illustrating an example of a structure for rotating the support plate.

FIG. 7 is a diagram illustrating an example of an ultrasonic probe, in which support plates are in a folded state.

FIG. 8 is a diagram illustrating an example of the ultrasonic probe of FIG. 7, in which the support plates are in an unfolded state.

FIG. 9 is a diagram illustrating an example of an ultrasonic probe, in which support plates are in a folded state; and

FIG. 10 is a diagram illustrating an example of the ultrasonic probe of FIG. 9, in which the support plates are in an unfolded state.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

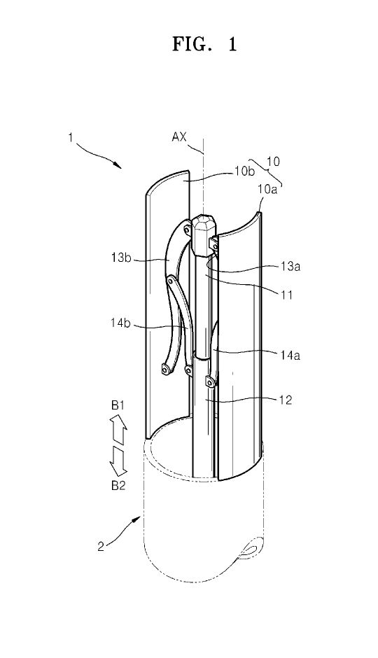

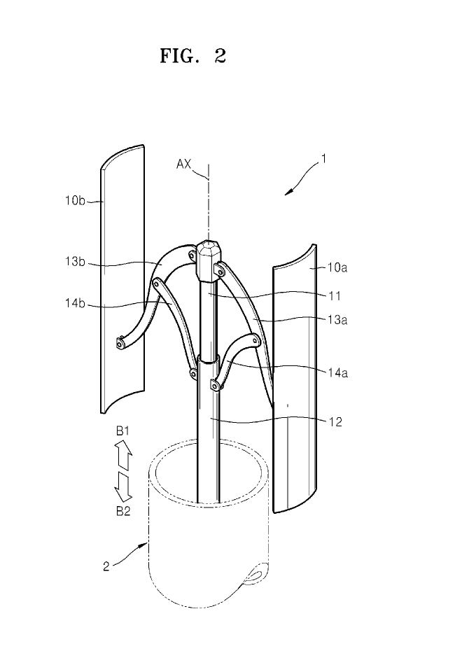

FIG. 1 is a diagram illustrating an example of an ultrasonic probe 1, in which a support plate 10 is in a folded state. FIG. 2 is a diagram illustrating an example of the ultrasonic probe 1 of FIG. 1, in which the support plate 10 is in an unfolded state.

Referring to FIGS. 1 and 2, the ultrasonic probe 1 is mounted on an end portion of a catheter 2 that is inserted in a tube-shaped organ of the human body. For example, tube-shaped organs in which the catheter 2 is inserted may include digestive intestines, respiratory organs, blood vessels, and other organs. When there is an affected part in the tube-shaped organ, the catheter 2 having the ultrasonic probe 1 mounted on the end portion thereof is inserted in the tube-shaped organ to allow the ultrasonic probe 1 to approach the position of the affected part. This allows the ultrasonic probe 1 to irradiate an ultrasound to the affected part, thereby generating a lesion. The lesion signifies that tissue of the affected part is locally destructed or necrosed. In some cases, whether a treatment is completed may be diagnosed by irradiating diagnostic ultrasound to the affected part and acquiring ultrasonic images. In other words, by using the ultrasonic probe 1, a treatment of the affected part of the tube-shaped organ is possible and a result of the treatment may be monitored by monitoring the state of the affected part.

The ultrasonic probe 1 is provided with at least one support plate 10 having a plurality of ultrasonic transducers 20. The support plate 10 has a first state in which the support plate 10 is folded and a second state in which the support plate 10 is unfolded for coupling to an inner wall of the tube-shaped organ. According to the above structure, the support plate 10 in the first state may be inserted in the tube-shaped organ, as illustrated in FIG. 1, changed to the second state, as illustrated in FIG. 2, after approaching the vicinity of the affected part. This allows the support plate 10 to be coupled to the inner wall of the tube-shaped organ. Thus, ultrasonic energy for diagnosis/treatment may be effectively transferred to the affected part.

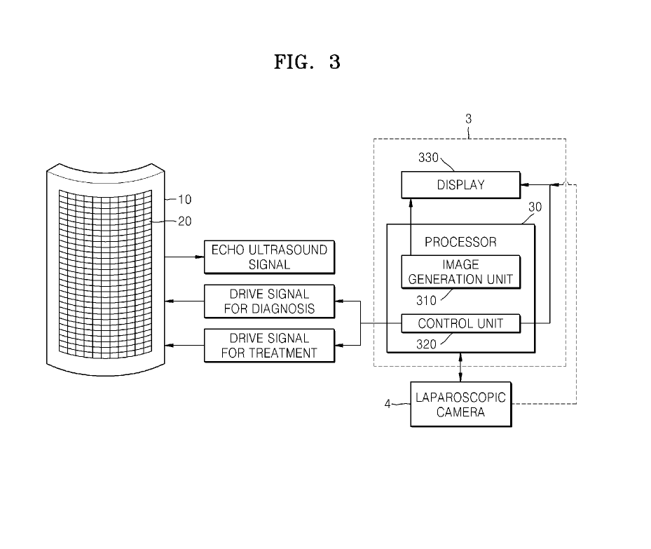

FIG. 3 is a diagram illustrating an example of an ultrasonic medical system adopting the ultrasonic probe 1. Referring to FIG. 3, ultrasonic transducers 20 may be arranged in an array form on the support plate 10. In this example, the ultrasonic transducers 20 convert external electrical signals into dynamic vibratory energy, thereby generating ultrasound, and convert external vibrations into electric signals. The ultrasonic transducers 20 may be transducers that generate high-intensity focused ultrasound (HIFU).

It should be appreciated that HIFU is capable of treating a lesion on a human body without using a knife or a needle and is harmless to the human body. HIFU treatment is used for tissue necrosis of a lesion by irradiating high-intensity ultrasound, which is about a hundred thousand times stronger than the intensity of ultrasound used for diagnosis. The high-intensity ultrasound is focused on a particular portion of the human body where the lesion is located and ultrasonic energy is irradiated. The ultrasonic energy is converted into thermal energy that increases the temperature around the irradiated portion and thus coagulation necrosis of the lesion tissue occurs. Since the temperature of the irradiated portion is instantly increased, heat is prevented from spreading to the surrounding region of the irradiated portion and only the irradiated portion is effectively removed. In this example, HIFU uses a transducer that receives an input of an electrical signal and generates ultrasound for irradiation. In an example, a super multi-element transducer may be used to obtain the HIFU treatment effect.

In an example where high-resolution operation may be necessary for diagnosis and treatment, the ultrasonic transducers 20 may be arranged in a 2D array. For example, capacitive micromachined ultrasonic transducers (cMUTs) may be employed as the ultrasonic transducers 20. While a piezoelectric micromachined ultrasonic transducer (pMUT) is difficult to manufacture in a micro size, a cMUT transducer is more easily manufactured and maintains a size that is merely several tens of microns.

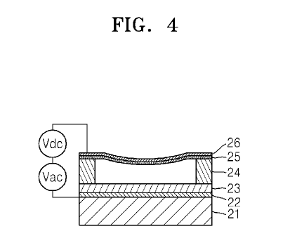

FIG. 4 is a diagram illustrating an example of a cMUT transducer unit. Referring to FIG. 4, the cMUT transducer is manufactured by forming a lower electrode 22, an insulating layer 23, and a pair of wall bodies 24 on a wafer 21. A diaphragm 25, on which an upper electrode 26 is deposited, is mounted across the wall bodies 24.

According to the above structure, the lower electrode 22 and the diaphragm 25 having the upper electrode 26 deposited thereon form a capacitor. When a direct voltage Vdc is applied between the lower and upper electrodes 22 and 26, the displacement of the diaphragm 25 occurs by an electrostatic force (Coulomb's force) and thus the diaphragm 25 is pulled toward the lower electrode 22. The displacement of the diaphragm 25 is stopped at a position where a resistance force due to internal stress and the electrostatic force are balanced. In this state, when an alternating voltage Vac that is smaller than the direct voltage Vdc is applied, the diaphragm 25 vibrates and ultrasound is generated.

Similarly, in a state in which direct voltage Vdc is applied and the diaphragm 25 is displaced, when an external ultrasonic pressure is applied to the diaphragm 25, the displacement of the diaphragm 25 is changed. The change in the displacement of the diaphragm 25 induces a change in capacitance. As the change in capacitance is detected, ultrasound may be received. In other words, by using the cMUT, the generation of ultrasound for treatment and the receiving of ultrasound for diagnosis is possible.

As illustrated in FIG. 4, since the cMUT may be manufactured by a series of semiconductor processes, several tens of thousands of ultrasonic transducers 20 may be arranged in a two dimensional area of several millimeters squared. Accordingly, compared to a case where pMUTs are used, a very high treatment accuracy may be achieved while simultaneously allowing for a high-resolution diagnostic images to be obtained during diagnosis.

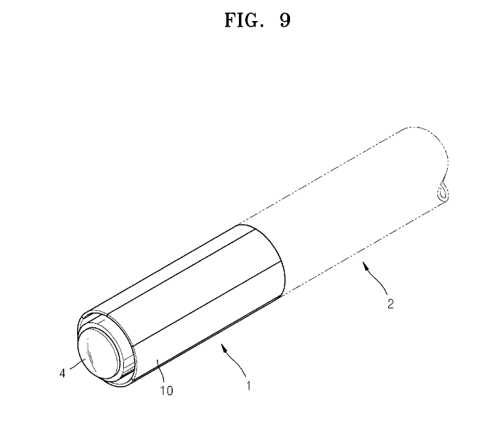

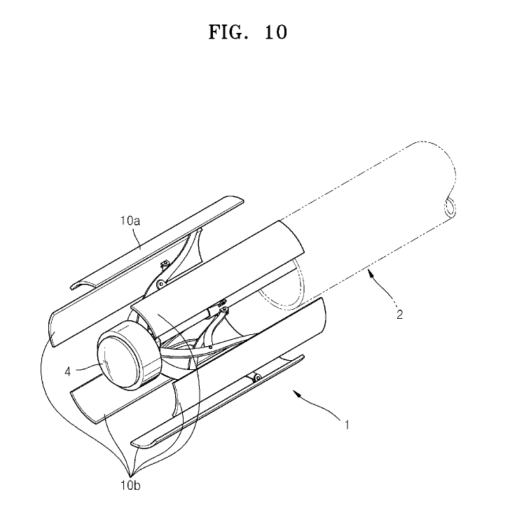

Referring back to the example illustrated in FIG. 3, the ultrasonic medical system includes a control station 3. In this example, the control station 3 includes a processor 30 for controlling the ultrasonic probe 1 and a display 330 for displaying an image. The control station 3 displays on the display 330 an image signal transferred from a laparoscopic camera 4. An endoscope image signal may be transferred to the display 330 directly or via a control unit 320. The laparoscopic camera 4 may be provided on the ultrasonic probe 1 as described later with reference to FIGS. 9 and 10.

In this example, the processor 30 includes an image generation unit 310 and a control unit 320. The processor 30 may be implemented by an array of a plurality of logic gates or by a combination of a common microprocessor and a memory for storing a program that is executable in the microprocessor. Also, should be appreciated by one of ordinary skill in the art that the processor 30 may be implemented by hardware in other appropriate forms.

In an example, the control unit 320 of the processor 30 generates a drive signal for treatment and a drive signal for diagnosis, if necessary. The control unit 320 determines the strength of irradiation on an affected part, the location of the irradiation on an affected part, and a focus area onto which a shear wave is to be guided. As a result of the determination, the control unit 320 controls the ultrasonic transducers 20. Also, the control unit 320 controls an irradiation time of each of the ultrasonic transducers 20. In addition, it should be appreciated by one of ordinary skill in the art that the control unit 320 can additionally control general operations of the ultrasonic probe 1.

For diagnosis, the ultrasonic transducers 20 generate echo ultrasonic signals by receiving echo ultrasounds reflected from an affected part. The image generation unit 310 receives the echo ultrasonic signals and generates ultrasonic images of the affected part by using the received echo ultrasonic signals. Since the general process of generating ultrasonic images by using echo ultrasonic signals is understood by one of ordinary skill in the art, a detailed description thereof will be omitted herein.

Referring back to the example illustrated in FIGS. 1 and 2, the support plate 10 includes first and second support plates 10a and 10b. The ultrasonic transducers 20 are provided on each of the first and second support plates 10a and 10b. In an example, the ultrasonic transducers 20 may be provided on only one of the first and second support plates 10a and 10b. In this case, the second support plate 10b where the ultrasonic transducers 20 are not provided is unfolded together with the first support plate 10a. Thereby, the second support plate 10b supports the inner wall of the tube-shaped organ so that the first support plate 10a may be stably coupled to the inner wall of the organ.

In this example, the first and second support plates 10a and 10b are symmetrically arranged about a center axis AX of the ultrasonic probe 1. The ultrasonic transducers 20 are connected to the processor 30 by an electric cable (not shown) that passes through the catheter 2. The processor 30 transmits a drive signal to drive the ultrasonic transducers 20 and receives a receiving signal by echo ultrasound via the electric cable. The ultrasonic transducers 20 and the processor 30 may be connected to each other in various methods, for example, a wireless method using a wireless transceiver.

Referring to FIGS. 1 and 2, the ultrasonic probe 1 is arranged at a leading end portion of the catheter 2. The ultrasonic probe 1 includes a first member 11 and a second member 12. At least a portion of the first member 11 is inserted in the second member 12. For example, as illustrated in FIG. 1, the first and second members 11 and 12 each have a tube shape.

In this example, the ultrasonic probe 1 includes first arms 13a and 13b and second arms 14a and 14b to fold/unfold the first and second support plates 10a and 10b. The first and second support plates 10a and 10b are connected to the first member 11 by the first arms 13a and 13b, respectively. One end portion of each of the first arms 13a and 13b is pivotally connected to the first member 11. The other end portions of the first arms 13a and 13b are pivotally connected to the first and second support plates 10a and 10b, respectively. One end portion of each of the second arms 14a and 14b is pivotally connected to the second member 12. The other end portions of the second arms 14a and 14b are pivotally connected to the first arms 13a and 13b, respectively, and between the end portions of each of the first arms 13a and 13b.

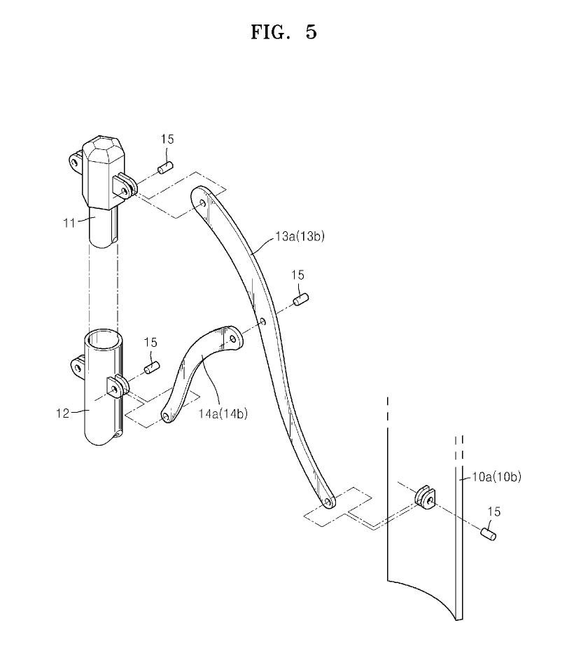

FIG. 5 is a diagram illustrating an example of a pivotal connection structure for folding/unfolding the support plate 10. Referring to FIG. 5, one end portion of each of the first arms 13a and 13b is pivotally connected to a leading end portion of the first member 11. For example, a pivot pin 15 functions as a pivot center axis into a hole formed in one end portion of each of the first arms 13a and 13b and a hole formed in the leading end portion of the first member 11.

Likewise, the other end portions of the first arms 13a and 13b are pivotally connected to the first and second support plates 10a and 10b. The one end portion of each of the second arms 14a and 14b is pivotally connected to the second member 12, and the other end portions of the second arms 14a and 14b is pivotally connected to the first arms 13a and 13b, respectively.

In this example, the first member 11 and the second member 12 may move relative to each other in a lengthwise direction. In other words, while the first member 11 is fixed, the second member 12 may move in a lengthwise direction with respect to the first member 11. Similarly, while the second member 12 is fixed, the first member 11 may move in a lengthwise direction with respect to the second member 12.

Referring to FIG. 1, when the second member 12 is pushed in a direction B1, the second arms 14a and 14b advance in the direction B1 and thus the first arms 13a and 13b are outwardly unfolded. As illustrated in FIG. 2, this results in the first and second support plates 10a and 10b being in an unfolded position. To fold the first and second support plates 10a and 10b, the second member 12 is pulled in a direction B2 of FIG. 2 and thus the first and second support plates 10a and 10b are folded.

To enable the above operation, the second member 12 is exposed to the outside through an end portion of the catheter 2 or connected to a manipulation mechanism (not shown) provided at the end portion of the catheter 2. This allows the second member 12 to be pushed or pulled by a catheter operator. The second member 12 may be connected to the manipulation mechanism directly or via a power transfer means such as a cable.

Alternatively, the first member 11 may move in a lengthwise direction with respect to the second member 12. Referring to FIG. 1, when the first member 11 is pulled in the direction B1, the first member 11 and the first arms 13a and 13b are pulled in the direction B2 and thus the first arms 13a and 13b and the second arms 14a and 14b are outwardly unfolded. As illustrated in FIG. 2, this results in the first and second support plates 10a and 10b being in an unfolded position. To fold the first and second support plates 10a and 10b, the first member 11 is pushed in the direction B1. To enable the above operation, the first member 11 is exposed to the outside through the end portion of the catheter 2 or connected to the manipulation mechanism provided at the end portion of the catheter 2. The first member 11 may be connected to the manipulation mechanism directly or via a power transfer means such as a cable.

As described above, since the first and second support plates 10a and 10b, each having a plate shape on which ultrasonic transducers 20 are arranged, are unfolded inside a tube-shaped organ, ultrasonic energy for diagnosis/treatment may be effectively transferred to an affected part. In an example, the first and second support plates 10a and 10b may have a curved plate shape. Also, since the first and second support plates 10a and 10b may be easily coupled to the inner wall of a tube-shaped organ by being unfolded, the ultrasonic probe 1 may be applied to organs having various sizes. That is, there is no need to have various ultrasonic probes having different thicknesses for applying to various organs based on the size or diameter of the organ.

After the ultrasonic probe 1 is inserted into the tube-shaped organ, the first support plate 10a on which the ultrasonic transducers 20 are arranged is moved toward an affected part. Thus, ultrasonic energy for diagnosis/treatment may be effectively transferred to the affected part. In an example, the first and second support plates 10a and 10b are rotatable inside the tube-shaped organ, individually or together, in the folded or unfolded position. For example, the first and second support plates 10a and 10b are rotated around the center axis AX of the ultrasonic probe 1. Accordingly, at least a part of the first member 11, for example, a part to which the first arms 13a and 13b connect, may be rotated. There may be various structures to rotate a part of the first member 11. In the following description, a rotation structure using a cable is described as an example thereof.

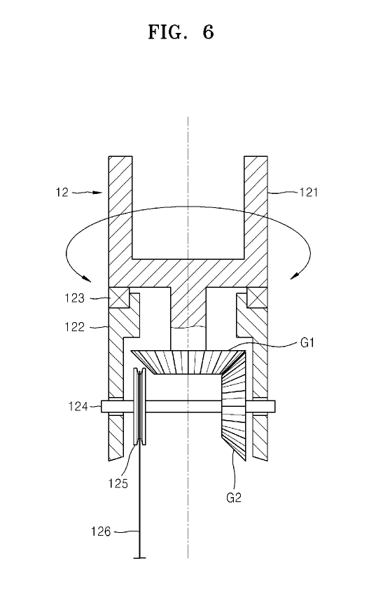

FIG. 6 is a diagram illustrating an example of a structure for rotating the first and second support plates 10a and 10b. Referring to FIG. 6, the second member 12 may include a rotating portion 121 and a fixed portion 122. The rotating portion 121 is supported on the fixed portion 122 with a bearing 123 interposed between the rotating portion 121 and the fixed portion 122. This allows the rotating portion 121 to rotate around the center axis AX. Although not illustrated in FIG. 6, an end portion of each the second arms 14a and 14b is pivotally connected to the rotating portion 121.

A first gear G1 having an axis that is coaxial with the center axis AX is provided at an end portion of the rotating portion 121. A second gear G2 that is engaged with the first gear G1 is provided in the fixed portion 122. In this example, The first gear G1 and the second gear G2 are bevel gears having axes that are perpendicular to each other. The second gear G2 is combined with a shaft 124 that is rotatably coupled to the fixed portion 122. A pulley 125 is provided on the shaft 124 with a cable 126 wound around the pulley 125.

In this example, the second gear G2 and the pulley 125 are fixed on the shaft 124 in order to allow rotation of the shaft 124. The pulley 125 is forwardly or backwardly rotated by selectively pulling one end of the cable 126 or the opposite end of the cable 126 using an actuator (not shown) or through manipulation by the catheter operator. When the cable is pulled, a rotation force is transferred to the first gear G1 via the second gear G2, thus the rotating portion 121 is rotated around the center axis AX. When the rotating portion 121 rotates, the first member 11 and the support plate 10 are rotated together because the first member 11 and support plate 10 are connected to the rotating portion 121 by the second arms 14a and 14b and the first arms 13a and 13b. As such, the first and second support plates 10a and 10b are capable of rotating to a position where ultrasonic energy may be effectively transferred to an affected part.

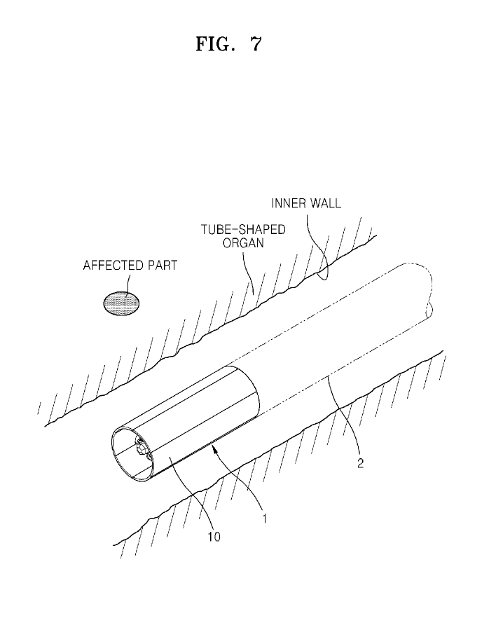

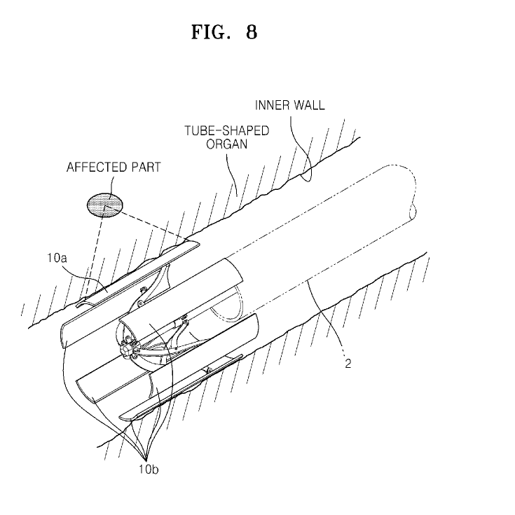

Although FIGS. 1 and 2 illustrate the ultrasonic probe 1 having two support plates, the present inventive concept is not limited thereto. The number of support plates 10 may be 3 or more. FIG. 7 is a diagram illustrating an example of an ultrasonic probe 1 in which support plates 10 are in a folded state. FIG. 8 is a diagram illustrating an example of the ultrasonic probe 1 of FIG. 7 in which the support plates 10 are in an unfolded state.

Referring to FIGS. 7 and 8, the ultrasonic probe 1 includes six support plates 10. At least one of the six support plates 10 may be a support plate 10a on which the ultrasonic transducers 20 are arranged, whereas the other support plates 10 may be support plates 10b having no ultrasonic transducers 20 thereon. In other examples, the number of support plates 10a and 10b are not limited to the number illustrated in this example, and the number of support plates 10a having ultrasonic transduces 20 may range from one to all of the support plates 10.

As illustrated in FIG. 7, the ultrasonic probe 1 having the support plates 10 in a folded state is inserted into a tube-shaped organ, for example, digestive intestines, respiratory organs, blood vessels, or other such organs. When the ultrasonic probe 1 approaches the vicinity of an affected part, the support plates 10 are unfolded. Since the process of unfolding the support plates 10 is the same as the process described with reference to FIGS. 1 and 2, repeated descriptions thereof are omitted. In this state, the support plates 10 may be rotated to allow the support plate 10a having the ultrasonic transducers 20 to face the affected part. The structure which enables rotation of the support plates 10 may be the same as the example of FIG. 6.

Referring to the example of FIG. 8 and also referring back to FIG. 3, when the support plates 10 are unfolded and coupled to the inner wall of the organ, the control unit 320 controls the ultrasonic transducers 20 to irradiate ultrasound for diagnosis. The ultrasonic transducers 20 receive echo ultrasound and the image generation unit 310 generates a visible image from the received echo ultrasound. While checking an image through the display 330, the catheter operator may operate the actuator or manipulate the cable 126 to rotate the support plate 10 in order to see an affected part. The catheter operator, may rotate the support plates 10 to allow the support plate 10a having the ultrasonic transducers 20 to face the affected part in an optimal state. The support plates 10 may be rotated in a state of being coupled to the inner wall of the organ. Also, the catheter operator may remove the coupling of the support plates 10 before rotating the support plate 10.

In an example, the support plate 10a may automatically move to face the affected part. For example, when the position of the affected part is not shown from an image generated by the image generation unit 310, the control unit 320 rotates the support plates 10 such that the clearest image of the affected part can be checked from the image. The control unit may rotate the support plates by driving the cables 126 using the actuator. If the position of the affected part is not manually checked from the image, a series of operations in which the control unit 320 folds the support plates 10, rotates the support plates 10 at a predetermined angle, and automatically checks the position of affected part from the image may be repeated. Accordingly, the first support plate 10a may be moved to face the affected part by a manual operation or an automation operation.

FIG. 9 is a diagram illustrating an example of an ultrasonic probe 1 in which support plates 10 are in a folded state. FIG. 10 is a diagram illustrating the ultrasonic probe 1 of FIG. 9 in which the support plates 10 are in an unfolded state. Referring to FIGS. 9 and 10, the ultrasonic probe 1 includes six support plates 10. At least one of the six support plates 10 is a support plate 10a on which the ultrasonic transducers 20 are arranged, whereas the other support plates 10 may be support plates 10b having no ultrasonic transducers 20 thereon. In this example, a laparoscopic camera 4 is mounted on a leading end portion of the first member 11. The laparoscopic camera 4 may have an illumination function. The laparoscopic camera 4 may be connected to the processor 30 or the display 330 of FIG. 3 by an electrical signal line passing through the inner space of the catheter 2.

In this example, a catheter operator inserts the catheter 2 with the ultrasonic probe 1, while checking the position of the affected part from an image transmitted by the laparoscopic camera 4, in order to control an insertion position of the ultrasonic probe 1. The catheter operator controls the ultrasonic probe 1 to approach the affected part through an insertion operation and then stops the insertion operation. As illustrated in FIGS. 7 and 8, the catheter operator may unfold the support plates 10, check an ultrasonic image, and rotate the support plates 10, so that the support plate 10a where the ultrasonic transducers 20 are arranged may be arranged to face the affected part. Accordingly, an affected part may be easily found and diagnosed/treated without a separate external imaging apparatus to check the position of the affected part.

The ultrasonic probe 1, the catheter 2, the control station 3, the laparoscopic camera 4, and all units described above may be implemented using one or more hardware components, or a combination of one or more hardware components and one or more software components. A hardware component may be, for example, a physical device that physically performs one or more operations, but is not limited thereto. Examples of hardware components include controllers, microphones, amplifiers, low-pass filters, high-pass filters, band-pass filters, analog-to-digital converters, digital-to-analog converters, and processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

Software or instructions for controlling a processing device to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An ultrasonic probe, comprising:
at least one support plate having a first state of being folded and a second state of being unfolded; and
ultrasonic transducers arranged on the at least one support plate.

2. The ultrasonic probe of claim 1, wherein
the at least one support plate comprises a plurality of support plates each having a first state of being folded and a second state of being unfolded;
the ultrasonic transducers are capacitive micromachined ultrasonic transducers which are arranged in an array on at least one of the plurality of support plates; and
the second states of the plurality of support plates allow the plurality of support plates to be coupled to an inner wall of an organ.

3. The ultrasonic probe of claim 2, further comprising:
a first member;
a second member capable of moving in a lengthwise direction with respect to the first member;
a plurality of first arms, each having one end connected to the first member and another end connected to one of the plurality of support plates; and
a plurality of second arms, each having one end connected to the second member and another end connected to one of the plurality of first arms at a position between the one end and the another end of the plurality of first arms, wherein
in response to the second member being moved with respect to the first member, the plurality of support plates are switched between the first states and the second states.

4. The ultrasonic probe of claim 3, wherein the first and second members are tube-shaped members and at least a part of the first member is inserted into the second member.

5. The ultrasonic probe of claim 3, further comprising a rotation unit for rotating the plurality of support plates around a center axis of the ultrasonic probe.

6. The ultrasonic probe of claim 5, wherein
the second member comprises a fixed portion and a rotating portion that is rotatably coupled to the fixed portion and to which the plurality of first arms are connected; and
the rotation unit rotates the rotating portion.

7. The ultrasonic probe of claim 5, wherein the rotation unit comprises:
a first gear provided at the rotating portion of the second member and having a rotation axis that is aligned with the center axis of the ultrasonic probe;
a second gear provided at the fixed portion of the second member and engaged with the first gear; and
a pulley provided at the fixed portion of the second member and configured to be driven by a cable to rotate the second gear.

8. The ultrasonic probe of claim 7, wherein the first and second gears are bevel gears.

9. The ultrasonic probe of claim 2, wherein the plurality of support plates comprise a first support plate on which the plurality of capacitive micromachined ultrasonic transducers are arranged and a second support plate on which the plurality of capacitive micromachined ultrasonic transducers are not arranged.

10. The ultrasonic probe of claim 9, wherein the plurality of support plates are symmetrically arranged about the center axis of the ultrasonic probe.

11. The ultrasonic probe of claim 1, further comprising a laparoscopic camera that is provided at a leading end portion of the ultrasonic probe.

12. An ultrasonic probe, comprising:
at least one support plate having a first state of being folded and a second state of being unfolded;
ultrasonic transducers arranged on the at least one support plate;
a first member on which the at least one support plate is supported; and
a laparoscopic camera provided at a leading end portion of the first member.

13. The ultrasonic probe of claim 12, wherein
the at least one support plate comprises a plurality of support plates each having a first state of being folded and a second state of being unfolded;
the ultrasonic transducers are arranged in a two-dimensional plane on at least one of the plurality of support plates; and
the second states of the plurality of support plates allow the plurality of support plates to be coupled to an inner wall of an organ.

14. The ultrasonic probe of claim 13, further comprising:
a second member capable of moving in a lengthwise direction with respect to the first member;
a plurality of first arms, each having one end connected to the first member and another end connected to one of the plurality of support plates; and
a plurality of second arms, each having one end connected to the second member and another end connected to one of the plurality of first arms at a position between the one end and the another end of the plurality of first arms.

15. The ultrasonic probe of claim 14, wherein
the second member comprises a fixed portion and a rotating portion that is rotatably coupled to the fixed portion and to which the plurality of first arms are connected;
the rotating portion is provided with a first gear;
the fixed portion is provided with a second gear that is engaged with the first gear and is provided with a pulley that is configured to be driven by a cable for rotating the second gear; and
the rotating portion is rotatable with respect to the fixed portion.

16. The ultrasonic probe of claim 13, wherein the plurality of support plates comprise a first support plate on which the plurality of ultrasonic transducers are arranged and a second support plate for supporting the ultrasonic probe on an inner wall of the organ along with the first support plate.

17. An ultrasonic medical system comprising:
the ultrasonic probe of claim 13; and
a controller for controlling the ultrasonic probe.

18. The ultrasonic medical system of claim 17, further comprising:
a second member capable of moving in a lengthwise direction with respect to the first member;
a plurality of first arms, each having one end connected to the first member and another end connected to one of the plurality of support plates; and
a plurality of second arms, each having one end connected to the second member and another end connected to one of the plurality of first arms at a position between the one end and the another end of the plurality of first arms.

19. The ultrasonic medical system of claim 18, wherein
the second member comprises a fixed portion and a rotating portion that is rotatably coupled to the fixed portion and to which the plurality of first arms are connected,
the rotating portion is provided with a first gear having a rotation axis aligned with a center axis of the ultrasonic probe,
the fixed portion is provided with a second gear that is engaged with the first gear and is provided with a pulley that is configured to be driven by a cable for rotating the second gear, and
the rotating portion is rotatable with respect to the fixed portion.

20. The ultrasonic medical system of claim 17, wherein the plurality of support plates comprise a first support plate on which the plurality of ultrasonic transducers are arranged and a second support plate for supporting the ultrasonic probe on an inner wall of the organ along with the first support plate.
